(19) United States
(12) Patent Application Publication
(10) Pub. No.: US 2014/0355745 A1
(43) Pub. Date: Dec. 4, 2014

(54) SEMICONDUCTOR RADIATION DETECTOR AND NUCLEAR MEDICINE DIAGNOSIS DEVICE

(71) Applicant: Hitachi, Ltd., Tokyo (JP)

(72) Inventor: Shinya Kominami, Tokyo (JP)

(73) Assignee: Hitachi, Ltd, Tokyo (JP)

(21) Appl. No.: 14/373,436

(22) PCT Filed: Jan. 25, 2013
(86) PCT No.: PCT/JP2013/051542

§ 371 Date: Jul. 21, 2014

(30) Foreign Application Priority Data
Jan. 27, 2012 (JP) 2012-014889

(51) Int. Cl. G01T 1/24; A61B 6/00; H01L 31/0224; H01L 31/032; H01L 31/08

(52) U.S. Cl. CPC G01T 1/244; H01L 31/032; H01L 31/085; H01L 31/0224; A61B 6/4258; A61B 6/4266
USPC 378/189; 257/76

(57) ABSTRACT

Provided are a thallium bromide semiconductor radiation detector having stable measurement performance with little noise increase even during prolonged measurement, and a nuclear medicine diagnosis device employing the same. In a semiconductor radiation detector using thallium bromide as a semiconductor crystal sandwiched between cathode and anode electrodes, a remaining surface, among surfaces of the semiconductor crystal, which is other than a surface covered with the cathode or anode electrode, is covered with a passivation layer including any one of two materials, that is, fluoride of thallium and chloride of thallium, or a mixture of any one of the two materials and bromide of thallium.

### TECHNICAL FIELD

The present invention relates to a semiconductor radiation detector and a nuclear medicine diagnosis device.

### BACKGROUND ART

In recent years, a nuclear medicine diagnosis device employing a radiation detector which measures radiation such as a γ-ray has become widespread. A typical nuclear medicine diagnosis device includes a gamma camera device, a single photon emission computed tomography (SPECT) imaging device and a positron emission tomography (PET) imaging device. Demand for the radiation detector has been increasing as one of countermeasures against dirty bomb terrorism, which is one of the subjects to be tackled considering homeland security.

A combination of a scintillator and a photomultiplier has been used in the related art for such a radiation detector. However, recent interest has focused on technology of a semiconductor radiation detector employing a semiconductor crystal such as cadmium telluride, cadmium zinc telluride, gallium arsenide, and thallium bromide.

The semiconductor radiation detector is configured to collect, into an electrode, electric charges which have been generated by an interaction between radiation and the semiconductor crystal, and to convert those electric charges into electric signals. Accordingly, in comparison with the radiation detector using the scintillator, the semiconductor radiation detector has various advantages, such as excellent efficiency of converting electric charges into electric signals and possibility of downsizing.

The semiconductor radiation detector includes, for example, a plate-like semiconductor crystal, a cathode electrode which is formed on one surface of the semiconductor crystal, and an anode electrode which faces the cathode electrode across the semiconductor crystal. A high direct current voltage is applied between these cathode and anode electrodes. Then, the semiconductor radiation detector draws, as signals from the cathode or anode electrode, electric charges generated when the semiconductor crystal is irradiated with radiation such as an X-ray and a γ-ray.

Especially, among the semiconductor crystals used in the semiconductor radiation detector, thallium bromide has a large linear attenuation coefficient resulted from a photoelectric effect, compared to other semiconductor crystals such as cadmium telluride, cadmium zinc telluride, and gallium arsenide. Further, thallium bromide has a γ-ray sensitivity equivalent to those of other semiconductor crystals even with a thin semiconductor crystal. As a result, a semiconductor radiation detector using thallium bromide and a nuclear medicine diagnosis device employing the semiconductor radiation detector can be downsized more than other semiconductor radiation detectors using semiconductor crystals other than thallium bromide, and other nuclear medicine diagnosis devices employing semiconductor radiation detectors other than thallium bromide.

Moreover, the thallium bromide semiconductor crystal is cheaper than other semiconductor crystals such as cadmium telluride, cadmium zinc telluride, and gallium arsenide. Accordingly, the semiconductor radiation detector using the thallium bromide semiconductor crystal and the nuclear medicine diagnosis device employing the semiconductor radiation detector are cheaper than other semiconductor radiation detectors and other nuclear medicine diagnosis devices using semiconductor radiation detectors other than thallium bromide.

In the semiconductor radiation detector using the thallium bromide semiconductor crystal, gold is used as a material for the cathode and anode electrodes (for example, see PTL 1, PTL 2 and NPL 1).

PTL 1 discloses semiconductor radiation detectors using cadmium telluride or cadmium zinc telluride as a semiconductor crystal. Such radiation detectors include one in which a passivation layer of an oxide of the semiconductor is formed on a side surface of the semiconductor crystal where no electrode is formed. Such radiation detectors also include one in which a plurality of rectangular electrodes is disposed on one surface of one semiconductor crystal and a passivation layer of an oxide of the semiconductor is formed in a gap between those electrodes.

PTL 2 discloses a semiconductor radiation detector in which a highly moisture-resistant insulating coating is applied on a side surface of a semiconductor crystal where no electrode is formed.

### CITATION LIST

### Patent Literature

PTL 1: US 2010/0032579 A1
PTL 2: US 2008/0149844 A1

### Non-Patent Literature

NPL 1: IEEE TRANSACTIONS ON NUCLEAR SCIENCE VOL. 56, No. 3, JUNE 2009 (see pp. 819 to 823)

### SUMMARY OF INVENTION

### Technical Problem

In the meantime, the semiconductor radiation detector using the thallium bromide semiconductor crystal or the nuclear medicine diagnosis device employing the semiconductor radiation detector is required to operate stably for a prolonged time. For example, the nuclear medicine diagnosis device is usually required to operate continuously for about eight hours during daytime in order to be available for medical services. Accordingly, the nuclear medicine diagnosis device in operation requires stabilized measurement performance of the semiconductor radiation detector. In other words, the nuclear medicine diagnosis device is required to stably measure an energy spectrum of an incident γ-ray.

However, when the present inventors actually produced semiconductor radiation detectors using the thallium bromide semiconductor crystal, and carried out continuous measurement for several hours, it turned out that noise gradually increased on a γ-ray energy spectrum and prevented many of the semiconductor radiation detectors from measuring stably.

The semiconductor radiation detector using thallium bromide includes a plate-like thallium bromide semiconductor crystal, a cathode electrode disposed on one surface of the semiconductor crystal, and an anode electrode disposed on the other surface of the semiconductor crystal. Among the surface of the thallium bromide semiconductor crystal, a part other than the part covered with the cathode and anode electrodes has the thallium bromide semiconductor crystal exposed as it is.

Accordingly, since there is a very little amount of thallium (metal) besides thallium bromide as impurities on a surface not covered with the cathode or anode electrode, it is considered that some of thallium reacts with oxygen in the air and forms thallium oxide. Electric resistivity of thallium bromide (hereinafter simply referred to as “resistivity”) is about 10¹⁰ Ω·cm, while resistivity of metallic thallium is as low as 2×10⁻⁵ Ω·cm. As thallium oxide, thallium oxide (I) (Tl₂O) and thallium oxide (II) (Tl₂O₃) exist. Although resistivity of thallium oxide (I) is unclear, bulk resistivity of thallium oxide (II) is 7×10⁻⁵ Ω·cm, which is significantly lower than that of thallium bromide. It is considered that thallium oxide (I) is gradually oxidized in the air to be converted into thallium oxide (II).

In cases where measurement is carried out with the semiconductor radiation detector using the thallium bromide semiconductor crystal, high direct current voltage of several hundred V is applied between the cathode and anode electrodes. However, continuous application of high voltage for a prolonged time is considered to cause a section having significantly lower resistivity than the thallium bromide crystal at a part not covered with the cathode or anode electrode, among the surface of the semiconductor crystal, and to cause a dark current between the cathode and anode electrodes to increase intermittently and randomly. As a result, noise increases on the energy spectrum, and energy resolution deteriorates so that many of the detectors have been estimated to be unable to measure stably.

Accordingly, there has been a problem in cases where the thallium bromide crystal is exposed as it is at a part not covered with the cathode or anode electrode among the surface of the thallium bromide semiconductor crystal in the semiconductor radiation detector. The problem is that the nuclear medicine diagnosis device using the thallium bromide semiconductor crystal in the semiconductor radiation detector cannot be stably used for a prolonged time, because it is highly possible that noise cannot be prevented from increasing when the semiconductor radiation detector is used for prolonged measurement.

The present invention is to solve the problem and an object of the present invention is to provide a semiconductor radiation detector, using a thallium bromide semiconductor crystal, which has little noise and stable measurement performance even for prolonged measurement, and a nuclear medicine diagnosis device employing the semiconductor radiation detector.

### Solution to Problem

In order to solve the problem, a first invention is a semiconductor radiation detector including a thallium bromide semiconductor crystal sandwiched between a cathode electrode and an anode electrode, wherein a remaining surface, among surfaces of the semiconductor crystal, which is other than a surface covered with the cathode or anode electrode, is covered with a passivation layer including any one of two materials, that is, fluoride of thallium or chloride of thallium, or including a mixture of any one of the two materials and bromide of thallium.

A moisture-resistant electrical insulating coating is desirably applied to the passivation layer.

According to the first invention, among the surfaces of the semiconductor crystal, a surface not covered with the cathode or anode electrode is covered with the passivation layer. Neither metallic thallium nor thallium oxide which have low resistivity do not exist in the passivation layer and in an interface between the thallium bromide included in the semiconductor crystal and the passivation layer. As a result, when prolonged measurement is carried out with the semiconductor radiation detector using the thallium bromide semiconductor crystal, a dark current between the cathode and anode electrodes can be prevented from increasing intermittently and randomly, and an energy spectrum can be measured stably.

A second invention is a nuclear medicine diagnosis device employing the semiconductor radiation detector of the first invention.

According to the second invention, there is provided a nuclear medicine diagnosis device capable of stably measuring an energy spectrum for a prolonged time and obtaining a clear image.

### Advantageous Effects of Invention

According to the present invention, there are provided a semiconductor radiation detector using a thallium bromide semiconductor crystal and having little noise and stable measurement performance during prolonged measurement, and a nuclear medicine diagnosis device employing the semiconductor radiation detector.

### BRIEF DESCRIPTION OF DRAWINGS

FIGS. 1(a) and 1(b) are schematic views showing a configuration of a semiconductor radiation detector according to a first embodiment, wherein FIG. 1(a) is a perspective view while FIG. 1(b) is a cross-sectional view.

FIG. 2 is a configuration diagram of a radiation detection circuit when radiation measurement is carried out with the semiconductor radiation detector according to the first embodiment.

FIG. 3 is a view for explaining time variation of a bias voltage applied to the semiconductor radiation detector according to the first embodiment.

FIGS. 4(a) and 4(b) are views for explaining a γ-ray energy spectrum of ⁵⁷Co source which is measured with the semiconductor radiation detector according to the first embodiment, wherein FIG. 4(a) is a view for explaining a γ-ray energy spectrum right after applying a bias voltage, while FIG. 4(b) is a view for explaining a γ-ray energy spectrum eight hours after onset of applying the bias voltage.

FIGS. 5(a) and 5(b) are schematic views showing a configuration of a semiconductor radiation detector according to a comparative example, wherein FIG. 5(a) is a perspective view while FIG. 5(b) is a cross-sectional view.

FIGS. 6(a) and 6(b) are views for explaining a γ-ray energy spectrum of ⁵⁷Co source which is measured by using the semiconductor radiation detector of the comparative example, wherein FIG. 6(a) is a view for explaining a γ-ray energy spectrum right after applying a bias voltage, while FIG. 6(b) is a view for explaining a γ-ray energy spectrum eight hours after onset of applying the bias voltage.

FIGS. 7(a) and 7(b) are schematic views showing a configuration of a semiconductor radiation detector according to a second embodiment, wherein FIG. 7(a) is a perspective view while FIG. 7(b) is a cross-sectional view.

FIG. 8 is a configuration diagram of a radiation detection circuit when radiation measurement is carried out with the semiconductor radiation detector according to the second embodiment.

FIGS. 9(a) and 9(b) are views for explaining a γ-ray energy spectrum of ⁵⁷Co source which is measured by using the semiconductor radiation detector of the second embodiment, wherein FIG. 9(a) is a view for explaining a γ-ray energy spectrum right after applying a bias voltage, while FIG. 9(b) is a view for explaining a γ-ray energy spectrum eight hours after onset of applying the bias voltage.

FIG. 10 is a schematic configuration view of a single photon emission computed tomography (SPECT) imaging device as a first example of application including the semiconductor radiation detector of the first and second embodiments in a nuclear medicine diagnosis device.

FIG. 11 is a schematic configuration view of a positron emission tomography (PET) imaging device as a second example of application including the semiconductor radiation detector of the first and second embodiments in a nuclear medicine diagnosis device.

### DESCRIPTION OF EMBODIMENTS

Hereinafter, a semiconductor radiation detector and a nuclear medicine diagnosis device employing the same according to the present invention will be described with reference to the drawings.

### Semiconductor Radiation Detector of First Embodiment

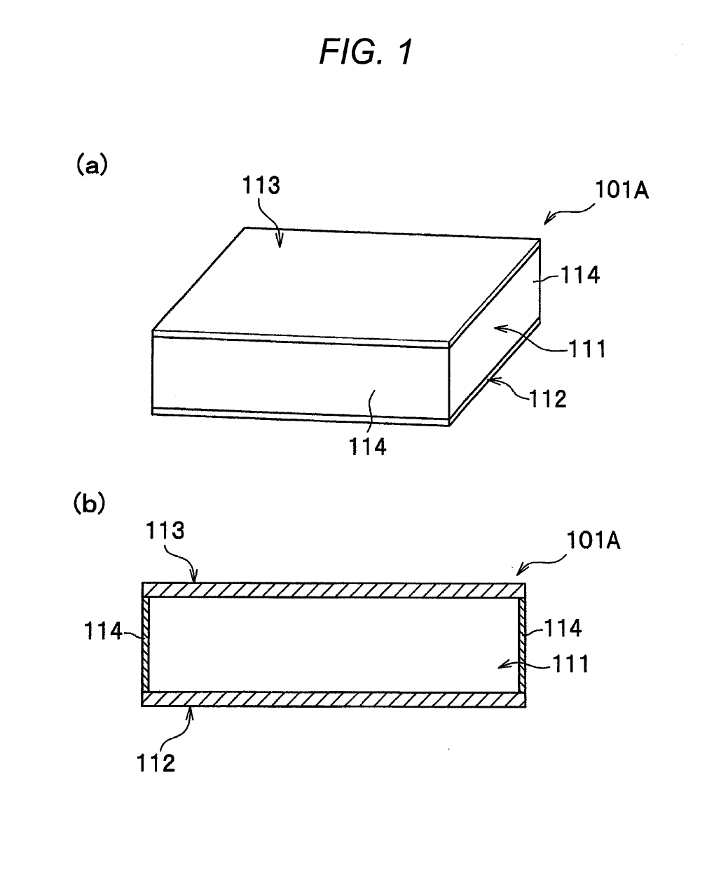

FIGS. 1(a) and 1(b) are schematic views showing a semiconductor radiation detector according to the first embodiment of the present invention. FIG. 1(a) is a perspective view, while FIG. 1(b) is a cross-sectional view.

As shown in FIGS. 1(a) and 1(b), a semiconductor radiation detector 101A of the present embodiment (hereinafter simply referred to as “detector 101A”) includes a plate-like semiconductor crystal 111, a first electrode (anode electrode, cathode electrode) 112 disposed on one surface of the semiconductor crystal 111 (under surface in FIGS. 1(a) and 1(b)), and a second electrode (cathode electrode, anode electrode) 113 disposed on the other surface of the crystal (upper surface in FIGS. 1(a) and (b)). Side passivation layers 114 are disposed on surfaces other than those covered with the first electrode 112 and the second electrode 113, among the surfaces of the semiconductor crystal 111. The side passivation layers 114 are disposed so as to cover the semiconductor crystal 111.

The side passivation layers 114 herein are called as such because the first and second electrodes 112, 113 are formed on two opposite surfaces of the semiconductor crystal 111. Accordingly, surfaces other than those covered with the first electrode 112 and the second electrode 113 mainly correspond to side portions. However, the “side passivation layers 114” are not restricted to the side portion as called. When there is a region on a part, among the two opposite surfaces of the semiconductor crystal 111, on which the first and second electrodes 112 and 113 are not formed, the “side passivation layers 114” also include that region.

The semiconductor crystal 111 is a region which generates electric charges by interacting with radiation (such as a γ-ray), and is formed by slicing a single crystal of thallium bromide (TlBr). In the present embodiment, the thickness of the semiconductor crystal 111 is, for example, 0.8 mm, while the width and depth of the surfaces on which the first and second electrodes 112, 113 are formed in FIG. 1(a) are, for example, 5.1 mm×5.0 mm. Thus, the semiconductor crystal 111 has a thin plate-like shape.

The first and second electrodes 112, 113 are formed from any one of gold, platinum, and palladium, and the thickness of each electrode is, for example, 50 nm (nanometers).

Each dimension of width and depth of the first and second electrodes 112, 113 in FIG. 1(a) is, for example, 5.1 mm×5.0 mm. The thickness of each side passivation layer 114 is, for example, about 8 nm.

Each dimension described above is illustrative only and is not restricted thereto, but the present embodiment will be described by using the dimension as an example.

Next, a process for producing the detector 101A including the semiconductor crystal 111, the first electrode 112, the second electrode 113, and the side passivation layers 114 will be described.

First, the first electrode 112 is formed by adhering, for example, 50 nm of gold, platinum or palladium, by means of an electron beam evaporation method, on one surface (under surface in FIGS. 1(a) and (b)) of the thallium bromide semiconductor crystal 111 formed into a plate-like shape in the dimension of, for example, 5.1 mm×5.0 mm.

Next, the second electrode 113 is formed by adhering 50 nm of gold, platinum or palladium, by means of the electron beam evaporation method, on the other surface of the semiconductor crystal 111 (upper surface in FIGS. 1(a) and (b)) opposite to the surface on which the first electrode 112 has been formed.

Then, the side passivation layers 114 including fluoride of thallium are formed by the following processes. That is, the whole surface is treated with fluorine plasma generated by high-frequency discharge of carbon tetrafluoride gas. This results in reduction of thallium oxide existing in surfaces, among the surfaces of the semiconductor crystal 111, which are not covered with the first electrode 112 or the second electrode 113 (corresponding to “a remaining surface, among the surfaces of the semiconductor crystal, which is other than those covered with the cathode or anode electrode” recited in claim). At the same time, generated thallium (metal) and thallium (metal) which has been generated near the surface during the production of the semiconductor crystal 111 are fluorinated. In such a case, the first and second electrodes 112, 113 include gold, platinum, or palladium. Accordingly, those electrodes do not react with the fluorine plasma and do not change.

Note that the side passivation layers 114 including fluoride of thallium are extremely thin. Therefore, there is a case where the side passivation layers 114 including fluoride of thallium are not formed on all surfaces, among the surfaces of the semiconductor crystal 111, which are not covered with the first electrode 112 or the second electrode 113 (corresponding to “a remaining surface, among the surfaces of the semiconductor crystal, which is other than those covered with the cathode or anode electrode” recited in claim). In such a case, thallium bromide included in the semiconductor crystal 111 is exposed locally so that the side passivation layers 114 form side passivation layers 114 including a mixture of fluoride of thallium and bromide of thallium.

Instead of treatment with the fluorine plasma, side passivation layers 114 including chloride of thallium may be formed by the following process. That is, the whole surface is treated with chlorine plasma generated by high-frequency discharge of boron trichloride gas. This results in reduction of thallium oxide existing in surfaces, among the surfaces of the semiconductor crystal 111, which are not covered with the first electrode 112 or the second electrode 113 (corresponding to “a remaining surface, among the surfaces of the semiconductor crystal, which is other than those covered with the cathode or anode electrode” recited in claim). At the same time, generated thallium (metal) and thallium generated near the surface during the production of the semiconductor crystal 111 are chlorinated. In such a case, the first and second electrodes 112, 113 include gold, platinum, or palladium. Accordingly, those electrodes do not react with the chlorine plasma and do not change.

Further, instead of treating with the fluorine plasma or the chlorine plasma, the side passivation layers 114 including chloride of thallium may be formed by the following process. That is, the whole surface is treated with hydrogen plasma generated by microwave discharge of hydrogen gas and steam gas. This results in reduction of thallium oxide existing in surfaces, among the surfaces of the semiconductor crystal 111, which are not covered with the first electrode 112 and/or the second electrode 113 (corresponding to “a remaining surface, among the surfaces of the semiconductor crystal, which is other than those covered with the cathode or anode electrode” recited in claim). After that, the semiconductor crystal 111 with the first and second electrodes 112, 113 is soaked into hydrochloric acid and chlorinated. In such a case, the first and second electrodes 112, 113 include gold, platinum, or palladium. Accordingly, those electrodes do not react with hydrogen plasma and hydrochloric acid, and do not change.

Note that the side passivation layers 114 including chloride of thallium, which are formed by treating the whole surface with chlorine plasma or by soaking into hydrochloric acid, are extremely thin. Therefore, there is a case where the side passivation layers 114 including chloride of thallium are not formed on all surfaces, among the surfaces of the semiconductor crystal 111, which are not covered with the first electrode 112 or the second electrode 113 (corresponding to “a remaining surface, among the surfaces of the semiconductor crystal, which is other than those covered with the cathode or anode electrode” recited in claim). In such a case, thallium bromide included in the semiconductor crystal 111 is exposed locally so that the side passivation layers 114 form side passivation layers 114 including a mixture of chloride of thallium and bromide of thallium.

The detector 101A is obtained by undergoing the following processes. That is, thallium oxide existing in surfaces, among the surfaces of the semiconductor crystal 111, which are not covered with the first electrode 112 or the second electrode 113 (corresponding to “a remaining surface, among the surfaces of the semiconductor crystal, which is other than those covered with the cathode or anode electrode” recited in claim) is reduced. At the same time, generated thallium (metal) and thallium (metal) generated near the surface during the production of the semiconductor crystal 111 are converted into fluoride of thallium or chloride of thallium. Then, side passivation layers 114 including fluoride of thallium, or side passivation layers 114 including a mixture of fluoride of thallium and bromide of thallium, or side passivation layers 114 including chloride of thallium, or side passivation layers 114 including a mixture of chloride of thallium and bromide of thallium are formed.

In the detector 101A according to the present embodiment, among the surfaces of the thallium bromide semiconductor crystal 111, surfaces not covered with the first electrode 112 or the second electrode 113 are covered with the side passivation layers 114 formed by fluorinating or chlorinating thallium. Accordingly, thallium bromide included in the semiconductor crystal 111 is not oxidized and the side passivation layers 114 themselves have sufficiently high resistivity compared to thallium (metal) and thallium oxide. Further, there is no residual thallium (metal) between the semiconductor crystal 111 and the side passivation layers 114.

### (Radiation Detection Circuit)

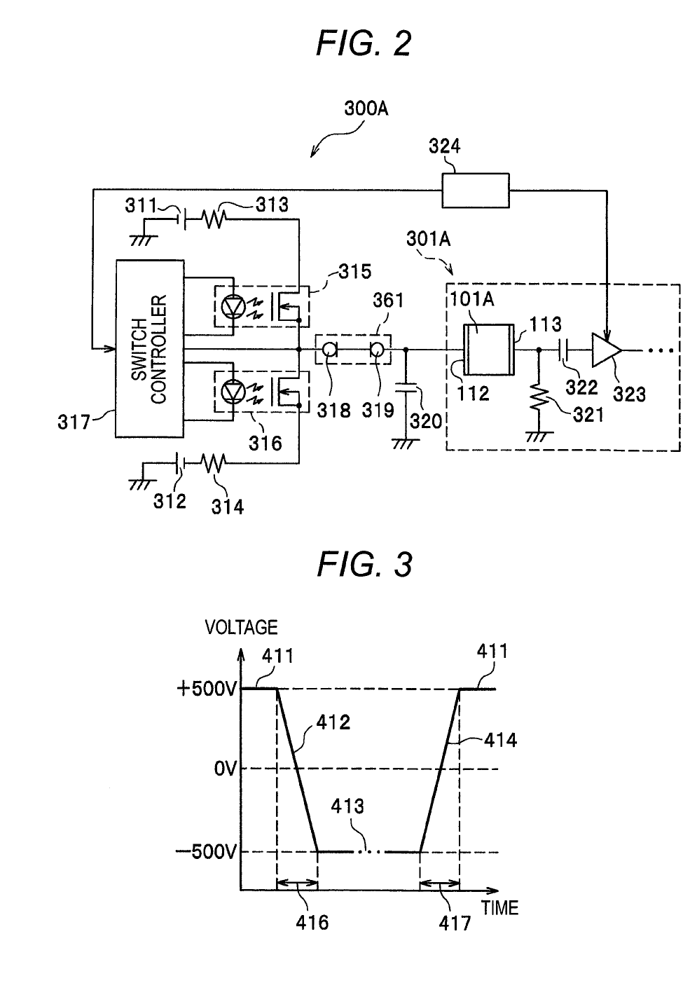

Next, a circuit configuration of a case where radiation measurement is carried out with the detector 101A will be described with reference to FIG. 2. FIG. 2 is a configuration diagram of a radiation detection circuit of a case where radiation measurement is carried out with the semiconductor radiation detector according to the first embodiment.

In FIG. 2, a radiation detection circuit 300A includes the detector 101A, a smoothing capacitor 320, a first direct current power source 311, and a second direct current power source 312. The detector 101A includes the semiconductor crystal 111 (see FIGS. 1(a) and (b)) and the first and second electrodes 112, 113 on two opposite surfaces of the semiconductor crystal 111. The smoothing capacitor 320 applies a voltage to the detector 101A. The first direct current power source 311 supplies positive charges to one electrode of the smoothing capacitor 320 (for example, to the side of the first electrode 112). The second direct current power source 312 supplies negative charges to the one electrode of the smoothing capacitor 320.

In FIG. 2, one electrode of the smoothing capacitor 320 is on the side of the first electrode 112, while the other electrode is on the side of a grounding wire. However, the configuration is not restricted to this example, and one electrode of the smoothing capacitor 320 may be on the side of the second electrode 113, and the other electrode may be on the side of the grounding wire.

The radiation detection circuit 300A further includes a first current regulative diode 318, a second current regulative diode 319, a first photoMOS relay 315, and a second photoMOS relay 316. The first current regulative diode 318, which is adjusted to have polarity coincident of a current regulative characteristic, is connected so as to apply current to the one electrode of the smoothing capacitor 320 from the first direct current power source 311. The second current regulative diode 319, which is adjusted to have polarity coincident of a current regulative characteristic, is connected so as to apply current to the second direct current power source 312 from the one electrode of the smoothing capacitor 320. The first photoMOS relay 315 is connected to a wire which connects the first direct current power source 311 and the one electrode of the smoothing capacitor 320. The second photoMOS relay 316 is connected to a wire which connects the second direct current power source 312 and the one electrode of the smoothing capacitor 320.

Herein, the first and second current regulative diodes 318, 319 constitute a current regulative device 361.

Further, as resistors for preventing overcurrent, a resistor 313 is disposed between the first direct current power source 311 and the first photoMOS relay 315, while a resistor 314 is disposed between the second direct current power source 312 and the second photoMOS relay 316.

A switch controller 317 controls opening and closing of the first and second photoMOS relays 315, 316.

The first and second photoMOS relays 315, 316 function as a relay (electric relay). Each photoMOS relay has high-speed responsiveness and high reliability, since it has no structural mechanical junction in order to prevent malfunction resulted from chattering or the like. That is why the photoMOS relay is employed herein.

One end of a bleeder resistor 321 and one electrode of a coupling capacitor 322 are connected to the output side of the detector 101A. An amplifier 323 which amplifies output signals of the detector 101A is connected to the other electrode of the coupling capacitor 322.

A negative electrode of the first direct current power source 311, a positive electrode of the second direct current power source 312, the other electrode of the smoothing capacitor 320, and the other end of the bleeder resistor 321 are each connected to the grounding wire.

Further, a polarity unifying controller 324 is connected to the switch controller 317 and the amplifier 323. The polarity unifying controller 324 controls opening/closing of the first and second photoMOS relays 315, 316, and the timing of output polarity inversion of the amplifier 323.

The first and second current regulative diodes 318, 319 mutually have opposite polarities of the current regulative characteristic, and are connected in series to configure the current regulative device 361. In this configuration, a typical current regulative diode presently used in the first and second current regulative diodes 318, 319 is configured to short-circuit a source electrode and a gate electrode of a field effect transistor (FET) to offer the current regulative characteristic. Accordingly, when a reverse voltage is applied to the current regulative diode, p-n junction formed in the FET is biased in the forward direction, so that a large current which applies a voltage to the first electrode 112 of the detector 101A flows. That is, a current characteristic of the current regulative diode has polarity.

Accordingly, the first and second current regulative diodes 318, 319 can be granted current regulative characteristic with no difference in polarity by reversing each polarity of current regulative characteristic and connecting the diodes in series. For this reason, the current regulative device 361 has current regulative characteristic with no difference in polarity, since the device includes the first and second current regulative diodes 318, 319 having opposite polarities of current regulative characteristic and being connected in series.

In measuring energy of radiation such as a γ-ray with the radiation detection circuit 300A, a bias voltage (e.g. +500 V or −500 V) for collecting electric charges is applied between the first and second electrodes 112, 113 of the detector 101A by means of the first or second direct current power source 311, 312 and the smoothing capacitor 320. When the detector 101A to which the bias voltage has been applied is irradiated with the γ-ray, an interaction occurs between the semiconductor crystal 111 (see FIGS. 1(a) and 1(b)) included in the detector 101A and the incident γ-ray, and electric charges such as electrons and holes are generated.

The bias voltage to be applied to the first electrode 112 of the detector 101A is switched between +500 V and −500 V as described above, for example. Accordingly, when a plus voltage is applied to the first electrode 112, the first electrode 112 becomes an anode electrode, while the second electrode 113 becomes a cathode electrode. In contrast, when a minus voltage is applied to the first electrode 112, the first electrode 112 becomes a cathode electrode, while the second electrode 113 becomes an anode electrode.

The generated electric charges are output as γ-ray detection signals (radiation detection signals) from the second electrode 113 of the detector 101A. This γ-ray detection signals are input to the amplifier 323 through the coupling capacitor 322. The bleeder resistor 321 prevents electric charges from continuously accumulating in the coupling capacitor 322, and functions to prevent an output voltage of the detector 101A from increasing too much. The amplifier 323 converts γ-ray detection signals which are minute electric charges into voltages, and amplifies the same.

The γ-ray detection signals amplified by the amplifier 323 are converted into digital signals by an analog/digital converter in a subsequent stage (not shown), and are counted by a data processing device (not shown) per γ-ray energy. The analog/digital converter in the subsequent stage and the device for processing γ-ray energy data are known in the related art, and are described, for example, in JP 2005-106807, and the details thereof will be omitted herein.

A part surrounded by broken lines and denoted with reference sign 301A in FIG. 2 represents a unit radiation detector circuit 301A which is provided to each detector 101A in a SPECT imaging device 600 and a PET imaging device 700 which are nuclear medicine diagnosis devices obtained by disposing a plurality of detectors 101A, as hereinafter described.

The amplifier 323 herein is of a type which can switch output polarity by the polarity unifying controller 324. That is, in detecting a γ-ray, the polarity unifying controller 324 controls the second electrode 113 of the detector 101A in FIG. 2 to collect negative charges or positive charges through the switch controller 317 and the first and second photoMOS relays 315, 316. Depending on the collected charges, the other electrode of the coupling capacitor 322 is controlled to output an output pulse having a positive voltage or an output pulse having a negative voltage.

Accordingly, the amplifier 323 is configured to have variable output polarity by command signals from the polarity unifying controller 324. For example, the amplifier 323 functions as a non-inverting amplifier when the other electrode of the coupling capacitor 322 outputs the output pulse having the positive voltage. On the other hand, the amplifier 323 functions as an inverting amplifier when the other electrode of the coupling capacitor 322 outputs the output pulse having the negative voltage.

The polarity unifying controller 324 transmits command signals such as “positive bias”, “negative bias”, “bias inversion from positive to negative”, and “bias inversion from negative to positive” to the switch controller 317 and the amplifier 323 based on, for example, previously installed polar inverse time information in every five minutes. The switch controller 317 opens/closes the first and second photoMOS relays 315, 316 based on the command signals

### (Polarization)

The semiconductor crystal 111 which is a member of the detector 101A (see FIGS. 1(a) and 1(b)) includes thallium bromide. Accordingly, for example, when a bias voltage of +500 V is continuously applied to the detector 101A using the first direct current power source 311, polarization (i.e. polarization in a structure and characteristic of the crystal) occurs in the semiconductor crystal 111. Further, radiation measurement performance is degenerated, and γ-ray energy resolution is deteriorated.

In order to prevent polarization, it is necessary to periodically invert the polarity of the bias voltage to be applied to the detector 101A. That is, it is necessary to invert polarity, for example, from +500 V to −500 V, and from −500 V to +500 V. Inversion is carried out in a cycle of, for example, 5 minutes.

First, a case where a bias voltage of +500 V is applied to the detector 101A will be described. A positive direct current bias voltage is supplied from the first direct current power source 311. When the voltage of +500 V is directly applied to the detector 101A from the first direct current power source 311, noise is generated. Accordingly, the grounded smoothing capacitor 320 is disposed between the detector 101A and the first direct current power source 311. Then, the voltage is applied to the first electrode 112 of the detector 101A. That is, the bias voltage to be applied to the detector 101A is substantially applied from the smoothing capacitor 320.

When a positive bias voltage is applied to the detector 101A, the switch controller 317 closes the first photoMOS relay 315 (the first photoMOS relay 315 is in on-mode), and also opens the second photoMOS relay 316 (the second photoMOS relay 316 is in off-mode).

The smoothing capacitor 320 is charged through the first current regulative diode 318 (and the second current regulative diode 319), and the voltage of the smoothing capacitor 320 becomes +500 V. As a result, the bias voltage to be applied to the detector 101A becomes +500 V as well.

In contrast, when a bias voltage of −500 V is applied to the detector 101A, the negative direct current bias voltage is supplied to the first electrode 112 of the detector 101A from the second direct current power source 312 mediated by the smoothing capacitor 320 which is grounded to suppress noise generation. When the negative bias voltage is applied to the detector 101A, the switch controller 317 opens the first photoMOS relay 315 (the first photoMOS relay 315 is in off-mode), and also closes the second photoMOS relay 316 (the second photoMOS relay 316 is in on-mode). The smoothing capacitor 320 is charged through the second current regulative diode 319 (and the first current regulative diode 318), and the voltage of the smoothing capacitor 320 becomes −500 V.

The radiation detection circuit 300A inverts positive and negative of the bias voltage to be applied to the detector 101A by accumulating positive charges or negative charges in one electrode of the smoothing capacitor 320.

Next, time variation of the bias voltage applied to the detector 101A will be described with reference to FIG. 3. FIG. 3 is a view for explaining time variation of the bias voltage applied to the semiconductor radiation detector according to the first embodiment. In the present embodiment, the bias voltage applied to the detector 101A is, for example, +500 V (reference sign 411) at first, and changes to −500 V (reference sign 413) by periodical inversion of the bias voltage afterward, and after five-minute duration, returns to +500 V (reference sign 411) again. The above-mentioned process is repeated afterward.

Time variation (reference signs 412, 414) straightly slopes during the bias voltage inversion, and this is the effect of the current regulative device 361. An absolute value of the bias voltage is insufficient for collecting electric charges during the bias voltage inversion, and γ-ray detection signals cannot be sufficiently output. However, each measurement downtime represented with reference signs 416, 417 is 0.3 seconds. During five-minute measurement, downtime of 0.3 seconds occurs. However, when the radiation detection circuit 300A is used as a nuclear medicine diagnosis device or a radiation detector for homeland security, 0.3 seconds is short enough and causes no problem.

### Radiation Measurement Performance of Semiconductor Radiation Detector According to First Embodiment

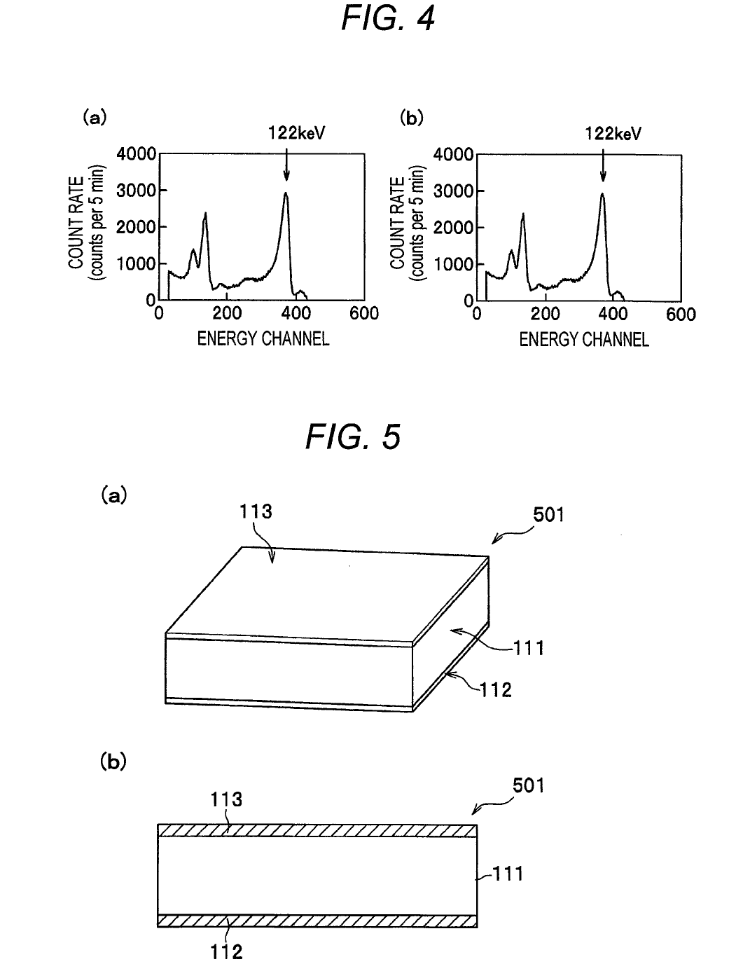

Next, radiation measurement performance of the detector 101A will be described with reference to FIGS. 4(a) and 4(b). FIGS. 4(a) and 4(b) are views for explaining a γ-ray energy spectrum of ⁵⁷Co source measured with the semiconductor radiation detector according to the first embodiment. FIG. 4(a) is a view for explaining a γ-ray energy spectrum right after applying a bias voltage, while FIG. 4(b) is a view for explaining a γ-ray energy spectrum eight hours after onset of applying the bias voltage. In FIGS. 4(a) and 4(b), the numbers of energy channel are taken along the abscissa. A pulse wave height of the γ-ray detection signals represents detected a γ-ray energy rate. Each number of an energy channel in FIGS. 4(a) and 4(b) shows each energy window (energy channel) in which a pulse wave height of γ-ray detection signals is set in a predetermined energy width by inputting the pulse wave height of the γ-ray detection signals into a multichannel wave height analyzer. Each number corresponds to a γ-ray energy rate represented by the γ-ray detection signals. For example, in FIG. 4(a), a γ-ray energy rate of almost 122 keV is allotted to the energy channel near almost the 370 channel. A count rate of the γ-ray energy channel (counts per 5 minutes) is taken along the ordinate.

In FIG. 4(a), the count rate of the energy channel corresponding to almost 122 keV reaches a peak. Energy resolution at the peak can be represented by the following formula.

Energy resolution=(the number of channels in half bandwidth of a peak)/(the number of channels right under the peak)

In the two views of FIGS. 4(a) and 4(b) showing the γ-ray energy spectra, each has energy resolution of almost 8% at 122 keV. In monitoring a dark current after operating the detector 101A of the present embodiment continuously for eight hours, the dark current remains about 0.1 μA and does not increase intermittently and randomly. Energy resolution remains almost 8% for at least eight hours without noise increase, and it is possible to stably measure radiation.

The above description is about the characteristic of the detector 101A when the side passivation layers 114 are disposed therein (see FIGS. 1(a) and 1(b)).

### Characteristic of Comparative Example with No Passivation Layer

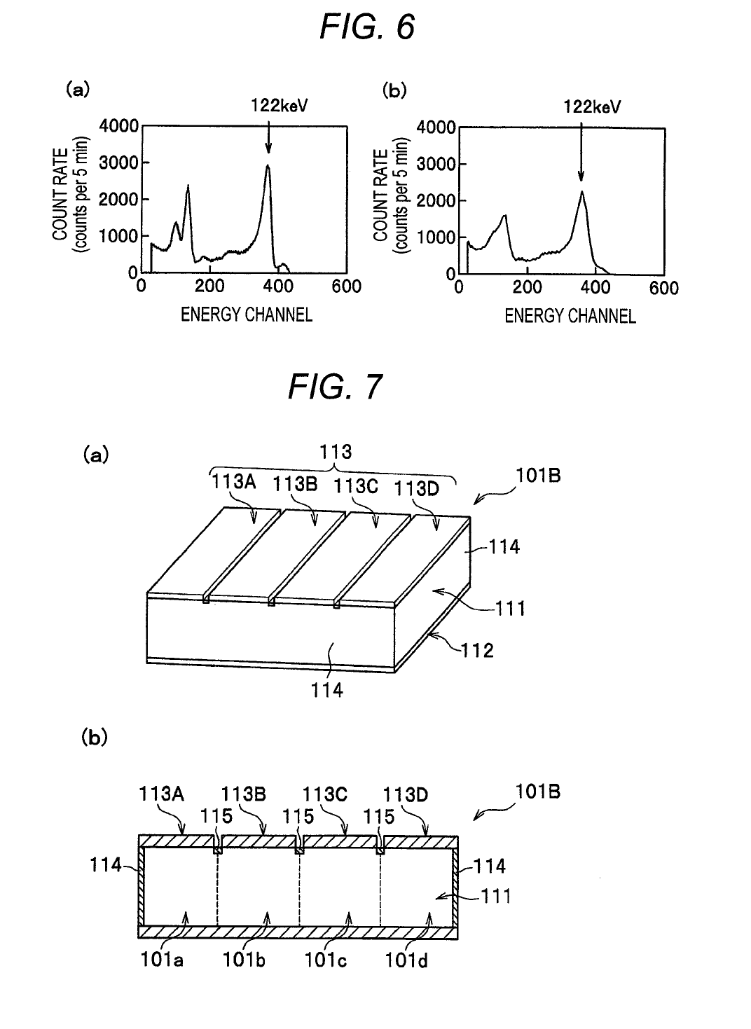

Next, a comparative example of a semiconductor detector 501 (hereinafter simply referred to as “detector 501”) with no side passivation layers 114 disposed therein will be described with reference to FIGS. 5(a), 5(b) and 6(a), 6(b). By comparing a characteristic thereof with that of FIGS. 4(a) and 4(b), distinction and superiority of the detector 101A with the side passivation layers 114 disposed therein will be described. FIGS. 5(a) and 5(b) are schematic views showing a configuration of a semiconductor radiation detector of the comparative example. FIG. 5(a) is a perspective view thereof, while FIG. 5(b) is a cross-sectional view thereof. FIGS. 6(a) and (b) are views for explaining a γ-ray energy spectrum of ⁵⁷Co source measured by using the semiconductor radiation detector of the comparative example. FIG. 6(a) is a view for explaining a γ-ray energy spectrum right after bias voltage application, while FIG. 6(b) is a view for explaining a γ-ray energy spectrum eight hours after onset of applying the bias voltage.

The comparative example shown in FIGS. 5(a) and 5(b) is a semiconductor detector with no passivation layer on a surface not covered with the first electrode 112 or the second electrode 113 of the thallium bromide semiconductor crystal 111.

Energy resolution at 122 keV in FIG. 6(a) is almost 8%. In contrast, energy resolution in FIG. 6(b) deteriorates into almost 12%. In monitoring a dark current between the first and second electrodes 112, 113 after operating the detector 501 of the comparative example continuously for eight hours, the dark current is about 0.12 μA right after applying a bias voltage, but changes intermittently and randomly between about 0.12 μA and 0.3 μA after eight hours.

In comparing the characteristic of the detector 101A of the first embodiment (see FIGS. 1(a) and (b)) with the characteristic of the detector 501 of the comparative example (see FIGS. 5(a) and 5(b)), the dark current does not increase even after operating continuously for eight hours and energy resolution does not change in the detector 101A of the first embodiment. On the other hand, in the detector 501 of the comparative example, the dark current increases intermittently and randomly after operating continuously for eight hours and energy resolution deteriorates greatly compared to energy resolution right after applying the bias.

Accordingly, the detector 101A of the first embodiment has been greatly improved from the viewpoint of stability in radiation measurement performance, compared to the detector 501 of the comparative example. This is an effect resulted from disposing the side passivation layers 114 in the detector 101A according to the first embodiment of the present invention.

### Semiconductor Radiation Detector of Second Embodiment

Next, a semiconductor radiation detector 101B according to a second embodiment of the present invention and a radiation detection circuit 300B employing the same will be described with reference to FIGS. 7(a) to 9(b).

The same components as those of the semiconductor radiation detector 101A of the first embodiment and the radiation detection circuit 300A thereof will be denoted with the same reference signs and the duplicate explanation thereof will be omitted herein.

FIGS. 7(a) and 7(b) are schematic views showing a configuration of the semiconductor radiation detector according to the second embodiment of the present invention. FIG. 7(a) is a perspective view, while FIG. 7(b) is a cross-sectional view.

As shown in FIG. 7(a), the semiconductor radiation detector 101B of the present embodiment (hereinafter simply referred to as “detector 101B”) includes a single semiconductor crystal 111, a first electrode (anode electrode, cathode electrode) 112 serving as a common electrode disposed on one surface of the semiconductor crystal 111 (under surface of FIGS. 7(a) and 7(b)), and a plurality of divided electrodes disposed on the other surface of the crystal, that is, for example, second electrodes (cathode electrodes, anode electrodes) 113A to 113D. The second electrodes 113A to 113D may be hereinafter simply referred to as the second electrode (anode electrode, cathode electrode) 113.

The side passivation layers 114 are formed on side surfaces, among the surfaces of the semiconductor crystal 111, which are other than the surfaces covered with the first electrode 112 or the second electrode 113. Further, inter-divided-electrode passivation layers 115 (see FIG. 7(b)) are formed in the gaps between the second electrodes 113A to 113D, which are disposed on the upper surface in FIGS. 7(a) and 7(b).

In one detector 101B of the present embodiment, the second electrode 113, which faces the common electrode, that is, the first electrode 112 across the semiconductor crystal 111, is divided into several divided electrodes. Accordingly, four detection units (channels) 101a to 101d are configured in total, which respectively correspond to the second electrodes 113A to 113D and function as individual semiconductor detectors (detection channels).

The semiconductor crystal 111 is a region which generates electric charges by interacting with radiation (such as a γ-ray), and is formed by slicing a single-crystal of thallium bromide (TlBr). In the present embodiment, the thickness of the semiconductor crystal 111 is, for example, 0.8 mm. The width and depth of the surfaces on which the first electrode 112 and the second electrodes 113A to 113D are formed in FIG. 7(a) are, for example, 5.1 mm×5.0 mm in a thin plate-like shape.

The first and second electrodes 112, 113 are formed from gold, platinum, or palladium. The thickness of each electrode is, for example, 50 nm.

The width and depth of the second electrodes 113A to 113D in FIG. 7(a) are, for example, 1.2 mm×5.0 mm.

Herein, each thickness of the side passivation layers 114 and the inter-divided-electrode passivation layers 115 is, for example, about 8 nm, and each width of the inter-divided-electrode passivation layers 115 in FIGS. 7(a) and 7(b) is, for example, 0.1 mm.

Each dimension described above is an example and is not restricted thereto. Further, the second electrode 113 may not be divided into four parts.

Next, a method for producing the detector 101B will be described, which includes the semiconductor crystal 111, the first electrode 112, the second electrodes 113A to 113D, the side passivation layers 114, and the inter-divided-electrode passivation layers 115.

First, the first electrode 112 is formed by adhering, for example, 50 nm of gold, platinum, or palladium, by means of an electron beam evaporation method, on one surface (under surface in FIG. 7(a)) of the thallium bromide semiconductor crystal ill formed in a plate-like shape.

Next, the second electrodes 113A to 113D which are the divided electrodes are formed on the other surface (upper surface in FIG. 7(a)) of the semiconductor crystal 111 opposite to the surface on which the first electrode 112 is formed, by the following processes. That is, photoresist is applied only to the gaps where the second electrodes 113A to 113D are not formed, and then, for example, 50 nm of gold, platinum, or palladium is adhered by means of the electron beam evaporation method.

Then, the side passivation layers 114 and the inter-divided-electrode passivation layers 115 including a passivation layer including fluoride of thallium or a mixture of fluoride of thallium and bromide of thallium are formed by the following processes. That is, the whole surface is treated with fluorine plasma generated by high-frequency discharge of carbon tetrafluoride gas, followed by reducing thallium oxide which exists on surfaces, among the surfaces of the semiconductor crystal 111, which are not covered with the first electrode 112 or the second electrodes 113A to 113D (corresponding to “a remaining surface, among the surfaces of the semiconductor crystal, which is other than those covered with the cathode or anode electrode” recited in claim). At the same time, generated thallium (metal) and thallium (metal) generated near the surface during the production of the semiconductor crystal 111 are fluorinated.

Instead of treatment with the fluorine plasma, the side passivation layers 114 and the inter-divided-electrode passivation layers 115 including a passivation layer including chloride of thallium or a mixture of chloride of thallium and bromide of thallium may be formed by the following processes. That is, the whole surface is treated with chlorine plasma generated by high-frequency discharge of boron trichloride gas, followed by reducing thallium oxide which exists on surfaces, among the surfaces of the semiconductor crystal 111, which are not covered with the first electrode 112 or the second electrodes 113A to 113D (corresponding to “a remaining surface, among the surfaces of the semiconductor crystal, which is other than those covered with the cathode or anode electrode” recited in claim). At the same time, the generated thallium (metal) and the thallium generated near the surface during the production of the semiconductor crystal 111 are chlorinated. In such a case, the first electrode 112 and the second electrodes 113A to 113D include gold, platinum, or palladium. Accordingly, those electrodes do not react with the fluorine plasma and do not change.

Further, instead of treating with the fluorine plasma or with the chlorine plasma, the side passivation layers 114 and the inter-divided-electrode passivation layers 115 including a passivation layer including chloride of thallium or a mixture of chloride of thallium and bromide of thallium may be formed by the following processes. That is, the whole surface is treated with hydrogen plasma generated by microwave discharge of hydrogen gas and steam gas, followed by reducing thallium oxide which exists on surfaces, among the surfaces of the semiconductor crystal 111, which are not covered with the first electrode 112 or the second electrodes 113A to 113D (corresponding to “a remaining surface, among the surfaces of the semiconductor crystal, which is other than those covered with the cathode or anode electrode” recited in claim). After that, the generated thallium (metal) and the thallium generated near the surface during the production of the semiconductor crystal 111 are chlorinated by being soaked in hydrochloric acid. In such a case, the first electrode 112 and the second electrodes 113A to 113D include gold, platinum, or palladium. Accordingly, those electrodes do not react with the hydrogen plasma or hydrochloric acid and do not change.

The detector 101B is obtained by carrying out the above processes. In the detector 101B of the present embodiment, among the surfaces of the thallium bromide semiconductor crystal 111, surfaces not covered with the first electrode 112 or the second electrodes 113A to 113D (corresponding to “a remaining surface, among the surfaces of the semiconductor crystal, which is other than those covered with the cathode or anode electrode” recited in claim) are covered with the side passivation layers 114 and the inter-divided-electrode passivation layers 115 formed by fluorinating or chlorinating thallium (metal). Accordingly, thallium bromide included in the semiconductor crystal 111 is not oxidized. Further, the side passivation layers 114 and the inter-divided-electrode passivation layers 115 themselves have sufficiently higher resistivity than thallium (metal) and oxide of thallium. Furthermore, thallium (metal) does not remain between the semiconductor crystal 111 and the side passivation layers 114 or the inter-divided-electrode passivation layers 115.

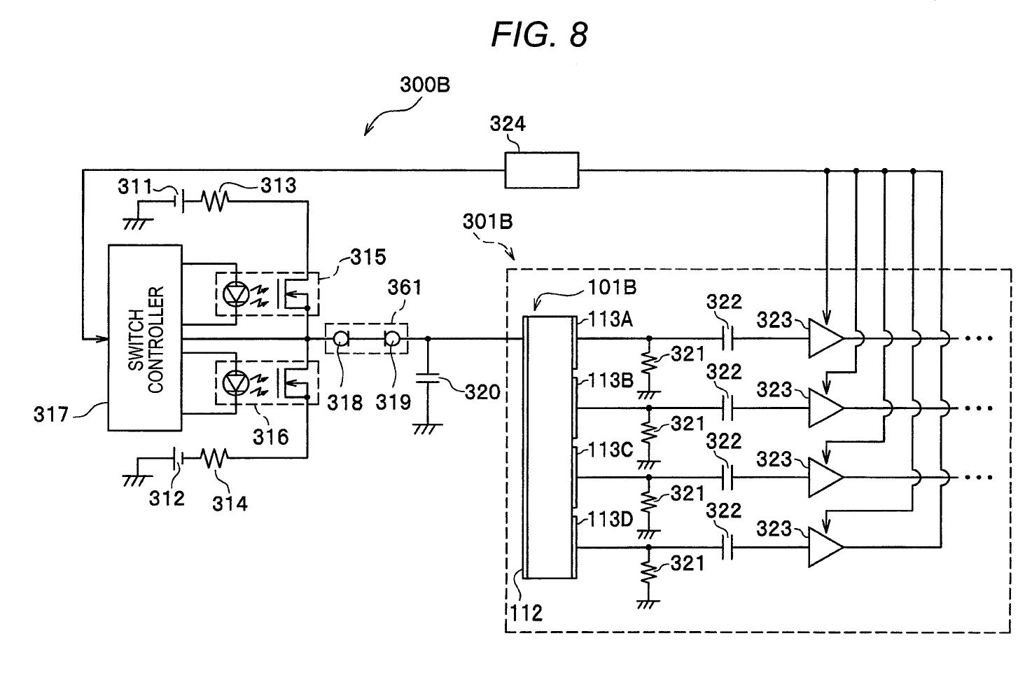

A circuit configuration for radiation measurement carried out with the detector 101B is almost the same as that of the radiation detection circuit 300A (see FIG. 2) for radiation measurement carried out with the detector 101A according to the first embodiment, and is shown in FIG. 8.

FIG. 8 is a configuration diagram of a radiation detection circuit when radiation measurement is carried out with the semiconductor radiation detector according to the second embodiment. A specific method for radiation measurement is exactly the same as the case described in the first embodiment (see FIG. 3).

The difference between the radiation detection circuit 300A shown in FIG. 2 and the radiation detection circuit 300B shown in FIG. 8 is that, in the radiation detection circuit 300B, each of the second electrodes 113A to 113D is provided with the bleeder resistor 321, the coupling capacitor 322, the amplifier 323, and the analog/digital converter (not shown) of the subsequent stage which processes output signals from the amplifier 323.

Each amplifier 323 receives command signals from the polarity unifying controller 324.

A part denoted with reference sign 301B and surrounded by broken lines in FIG. 8 represents a unit radiation detector circuit 301B. The unit radiation detector circuit 301B is disposed in each detector 101B of the SPECT imaging device 600 or the PET imaging device 700 to be hereinafter described, which is the nuclear medicine diagnosis device including a plurality of the detectors 101B.

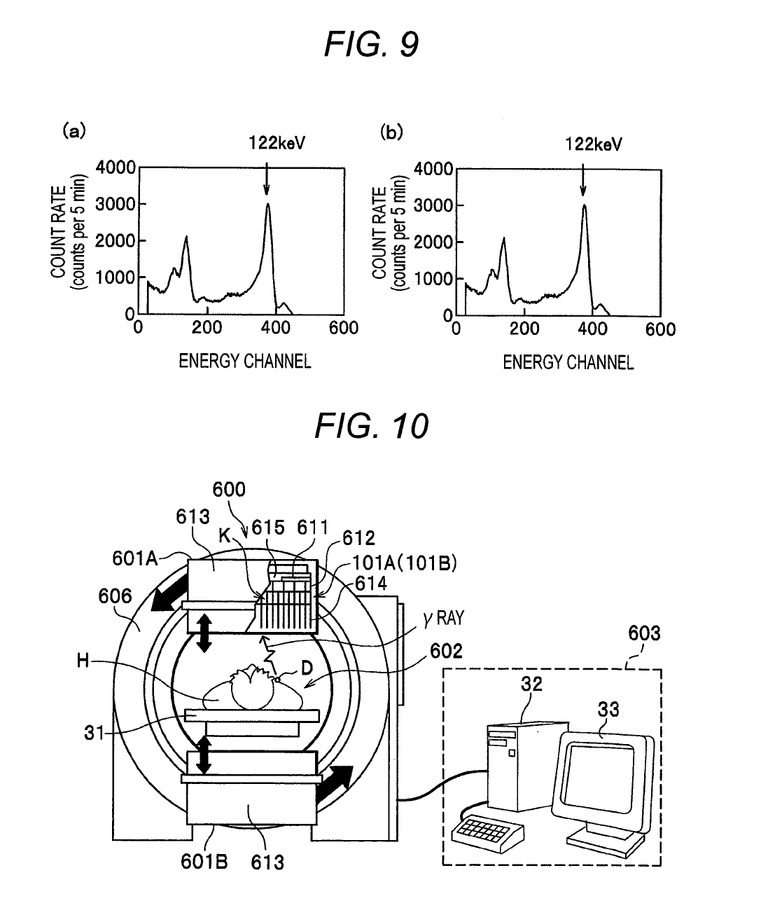

FIGS. 9(a) and 9(b) are views for explaining a γ-ray energy spectrum of ⁵⁷Co source measured with the semiconductor radiation detector of the second embodiment. FIG. 9(a) is a view for explaining a γ-ray energy spectrum right after bias voltage application, while FIG. 9(b) is a view for explaining a γ-ray energy spectrum eight hours after onset of applying the bias voltage.

FIGS. 9(a) and 9(b) are γ-ray energy spectra of ⁵⁷Co source measured with the detection unit 101a (see FIG. 7(b)) in the detector 101B according to the present embodiment. In other words, those views are γ-ray energy spectra of ⁵⁷Co source measured with the first electrode 112 and the second electrode 113A. Each energy resolution at 122 keV in these two views, FIGS. 9(a) and 9(b), is almost 7%. In cases where the detection units 101b to 101d are employed, energy resolution is exactly the same. In monitoring a dark current between the first electrode 112 and the second electrodes 113A to 113D while operating the detector 101B of the present embodiment continuously for eight hours, each dark current remains about 0.03 μA, and does not increase intermittently and randomly. All the four detection units 101a to 101d maintain energy resolution of almost 7% without noise increase for at least eight hours, and it is possible to stably measure radiation.

### Other Embodiment

The side passivation layers 114 in the detector 101A of the first embodiment, and the side passivation layers 114 and the inter-divided-electrode passivation layers 115 in the detector 101B of the second embodiment include any one of fluoride of thallium, chloride of thallium, a mixture of fluoride of thallium and bromide of thallium, and a mixture of chloride of thallium and bromide of thallium.

However, examples of fluoride of thallium generated by treatment with the fluorine plasma may include TlF and TlF₃. Examples of chloride of thallium generated by treatment with the chlorine plasma or by treating the whole surface with hydrogen plasma and then soaking into hydrochloric acid include TlCl, Tl₂Cl₃, TlCl₂, and TlCl₄.

Such fluoride of thallium and chloride of thallium include one that absorbs moisture in the air and converts its compound composition.

In order to prevent the side passivation layers 114 and the inter-divided-electrode passivation layers 115 from absorbing moisture in the air and converting each property, the side passivation layers 114 and the inter-divided-electrode passivation layers 115 may have high stability by coating at least those layers 114, 115 with a moisture-resistant insulating coating, for example, HumiSeal (registered trademark of Chase Corp.). In such a case, the first and second electrodes 112, 113 may be coated with the moisture-resistant insulating coating together with the side passivation layers 114 and the inter-divided-electrode passivation layers 115.

In the radiation detection circuit 300A in FIG. 2 and the radiation detection circuit 300B in FIG. 8, the first current regulative diode 318 and the second current regulative diode 319 are used while connected in series, but three or more current regulative diodes may be combined as well. Other devices and circuits may be used as long as they show the current regulative characteristic.

Further, an example of using the first and second photoMOS relays 315, 316 in the radiation detection circuit 300A in FIG. 2 and in the radiation detection circuit 300B in FIG. 8 has been described. However, it may not necessarily be a photoMOS relay, since the function of those photoMOS relays is a relay. A general relay can be used as long as it ensures reliability.

### First Example of Applying Detectors 101A, 101B According to First and Second Embodiments to Nuclear Medicine Diagnosis Device

The above-described semiconductor radiation detector (detector) 101A of the first embodiment and the semiconductor radiation detector (detector) 101B of the second embodiment can be applied to a nuclear medicine diagnosis device. FIG. 10 is a schematic configuration view of a single photon emission computed tomography (SPECT) imaging device as a first example of applying the detectors according to the first and second embodiments to a nuclear medicine diagnosis device.

FIG. 10 is a schematic configuration view showing the detector 101A of the first embodiment or the detector 101B of the second embodiment applied to the SPECT imaging device 600 as a nuclear medicine diagnosis device. The SPECT imaging device 600 in FIG. 10 includes, for example, two radiation detection blocks (camera units) 601A and 601B disposed in opposing positions, a rotative supporter (camera revolving pedestal) 606, a bed 31, and an image information creating device 603, so as to encircle a hollow cylindrical measurement region 602 in the center.

Herein, the two radiation detection blocks 601A, 601B have the same configuration. The configuration will be described with an example of the radiation detection block 601A located upside of FIG. 10. The radiation detection block 601A includes a plurality of radiation measurement units 611, a unit supporting member 615, and a light/electromagnetic shield 613. The radiation measurement units 611 include an interconnection substrate 612 in which a plurality of detectors 101A (or 101B) is mounted in a predetermined arrangement, and a collimator 614.

The image information creating device 603 includes a data processing device 32 and a display 33.

The radiation detection blocks 601A and 601B are disposed in the rotative supporter 606, for example, at positions different by 180 degrees in a circumferential direction. Specifically, each unit supporting member 615 (only the radiation detection block 601A is illustrated in a partial cross-sectional view) of the radiation detection blocks 601A and 601B is attached to the rotative supporter 606 so that the radiation detection blocks 601A and 601B are placed at positions different by 180 degrees in the circumferential direction. A plurality of the radiation measurement units 611 including the interconnection substrate 612 is removably attached to the unit supporting members 615.

A plurality of the detectors 101A (101B) is attached to the interconnection substrate 612 in a region K separated with the collimator 614, and is disposed in multistage of the collimator 614 so as to correspond to, for example, a plurality of radiation passages arranged in a two-dimensional surface. The collimator 614 is formed of a radiation shielding material such as lead and tungsten, and forms a plurality of radiation passages through which radiation, for example, a γ-ray passes.

All the interconnection substrate 612 and collimator 614 are disposed inside the light/electromagnetic shield 613 installed in the rotative supporter 606. The light/electromagnetic shield 613 allows a γ-ray to pass therethrough and shields any influence of electromagnetic waves other than a γ-ray on the detector 101A (101B) and the like.

In such a SPECT imaging device 600, a subject H to whom a radioactive pharmaceutical has been administered is placed on the bed 31. The bed 31 is moved, whereby the subject H is moved to the measurement region 602. As the rotative supporter 606 rotates, each of the radiation detection blocks 601A and 601B revolves around the subject H. Then, a γ-ray emitted from the radioactive pharmaceutical inside the subject H is detected.

When a γ-ray is emitted from an accumulation section D (e.g. affected area) inside the subject H, where the radioactive pharmaceutical is accumulated, the emitted γ-ray passes through the radiation passages of the collimator 614 and enters the detector 101A (101B) disposed in accordance with each radiation passage. The detector 101A (101B) then outputs γ-ray detection signals (radiation detection signals). The γ-ray detection signals are counted by the data processing device 32 per γ-ray energy (per energy channel), and then, information and the like of that count is displayed on the display 33.

In FIG. 10, the radiation detection blocks 601A, 601B rotate while being supported by the rotative supporter 606, as shown with thick arrows, and carry out imaging as well as measurement while changing the angle between the blocks and the subject H. As shown with thin arrows, the radiation detection blocks 601A, 601B are movable in a radially outward direction and in a radially inward direction from an axis of the hollow cylindrical measurement region 602. Therefore, the radiation detection blocks 601A, 601B can change the distance from the subject H.

The detector 101A (101B) employed in such a SPECT imaging device 600 uses thallium bromide of the semiconductor crystal 111 in which the side passivation layers 114 (the side passivation layers 114 and the inter-divided-electrode passivation layers 115 in the detector 101B) are formed on parts not covered with the first and second electrodes 112, 113. The detector 101A (101B) inverts, at a predetermined time interval, positive and negative of the bias voltage for collecting electric charges to be applied to the detector 101A (101B), and uses the bias voltage in order to prevent polarization. As a result, even though the detector 101A (101B) is used for prolonged measurement, the detector has stable energy resolution as well as a stable and little dark current. Therefore, the detector 101A (101B) is granted stable radiation measurement performance with little noise increase. Accordingly, the SPECT imaging device 600 which is downsized, cheap and stably operable for a consecutive long time can be provided.

As described above, the detectors 101A and 101B of the first and second embodiments are not restricted to the SPECT imaging device 600, but can be used for a gamma camera device, a PET imaging device or the like as a nuclear medicine diagnosis device. Next, an example of application to a PET imaging device will be described.

### Second Example of Applying Semiconductor Radiation Detector of Present Embodiment to Nuclear Medicine Diagnosis Device

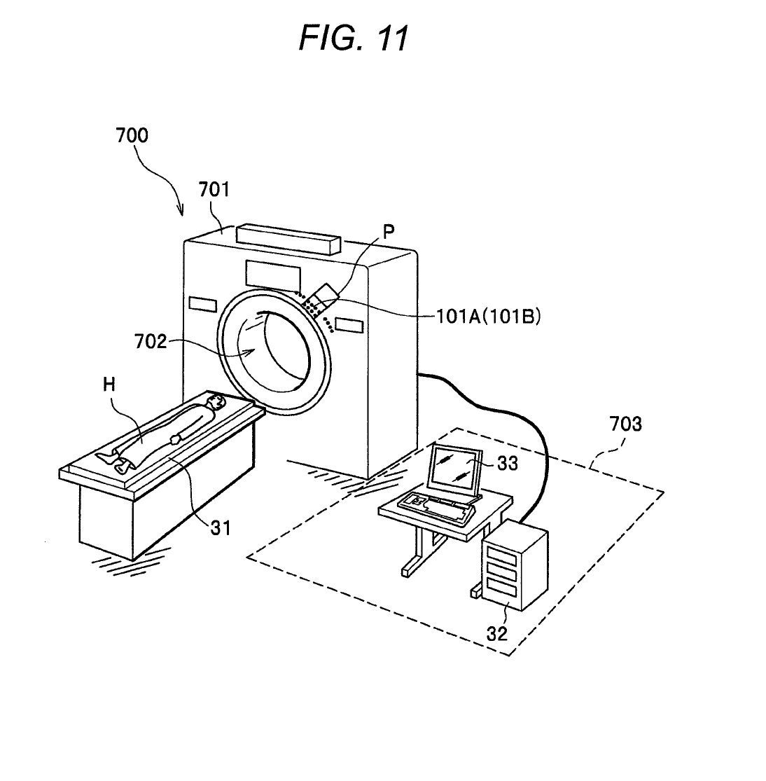

FIG. 11 is a schematic configuration view of a positron emission tomography imaging device (PET imaging device) as a second example of application including the semiconductor radiation detector according to the first and second embodiments in a nuclear medicine diagnosis device.

In FIG. 11, this PET imaging device (nuclear medicine diagnosis device) 700 includes an imaging device 701 having a hollow cylindrical measurement region 702 in the center, a bed 31 which supports the subject H and is movable in a longitudinal direction, and an image information creating device 703.

The image information creating device 703 includes a data processing device 32 and a display 33.

In the imaging device (camera unit) 701, a plurality of printed substrates (interconnection substrates) P, on each of which a plurality of the detectors 101A (or the detectors 101B) is mounted, is disposed in a circumferential direction so as to encircle the measurement region 702.

Such a PET imaging device 700 includes, for example, a digital application specific integrated circuit (ASIC, for a digital circuit, not shown) having data processing functionality. In the PET imaging device, a packet is produced, which has a γ-ray energy rate determined from γ-ray detection signals (radiation detection signals), detection time, and a detection channel identification (ID) of the detector 101A (101B). The produced packet is input to the data processing device 32.

Note that, when the detector 101B is used, the detection units (channel) 101a to 101d respectively constitute individual detection channels, each of which is granted a detection channel ID.

During examination, the γ-ray emitted from inside the body of the subject H due to the radioactive pharmaceutical is detected by the detector 101A (101B). That is, when a positron emitted from the radioactive pharmaceutical for the PET imaging disappears, a pair of γ-rays is emitted in directions different by substantially 180 degrees, and detected by separate detection channels ID among a plurality of the detectors 101A (101B). The detected γ-ray detection signals are input to the digital ASIC and the signals are processed as described above. Then, a γ-ray energy rate determined from γ-ray detection signals, position information of a detection channel which has detected a γ-ray (position information of the detection channel is stored beforehand in accordance with the detection channel ID), and γ-ray detection time information are input to the data processing device 32.

With the data processing device 32, a pair of γ-rays which has been generated after one positron disappears is counted as one (coincidence counting), and positions of the two detection channels which have detected the pair of γ-rays are specified based on the position information. The data processing device 32 creates tomography information (image information) of the radioactive pharmaceutical accumulating position, that is, a tumor position in the subject H by using the measured value obtained from coincidence counting and position information of the detection channel. The tomography image is displayed on the display 33.

The detector 101A (101B) employed in such a PET imaging device 700 uses thallium bromide of the semiconductor crystal 111 in which the side passivation layers 114 (the side passivation layers 114 and the inter-divided-electrode passivation layers 115 in the detector 101B) are formed on parts not covered with the first and second electrodes 112, 113. The detector 101A (101B) inverts, at a predetermined time interval, positive and negative of a bias voltage for collecting electric charges to be applied to the detector 101A (101B), and uses the bias voltage in order to prevent polarization. As a result, even though the detector 101A (101B) is used for prolonged measurement, the detector has stable energy resolution as well as a stable and little dark current. Therefore, the detector 101A (101B) is granted stable radiation measurement performance with little noise increase. Accordingly, the PET imaging device 700 which is downsized, cheap and stably operable for a consecutive long time can be provided.

As described above, according to the present invention, while using thallium bromide as a semiconductor crystal included in a radiation detector, stable measurement performance with little noise increase can be obtained even though the radiation detector is used for prolonged measurement. Accordingly, a semiconductor radiation detector which is downsized, cheap and operable for a prolonged time with stable performance as well as a nuclear medicine diagnosis device in which the semiconductor radiation detector is mounted can be provided.

In the nuclear medicine diagnosis device such as the SPECT imaging device 600 and the PET imaging device 700, the data processing device 32 and the display 33 have been exemplified as the image information creating devices 603, 703 shown in FIGS. 10 and 11. However, an image information creating device may not, be a combination of the data processing device 32 and the display 33, since there are various modes of data processing.

### INDUSTRIAL APPLICABILITY

According to the present invention, the semiconductor radiation detectors 101A, 101B, and the nuclear medicine diagnosis devices 600, 700 including the same can be downsized and reduce costs while securing a stable operation of these nuclear medicine diagnosis devices. Accordingly, it is possible that these detectors and devices will contribute to prevalence of the nuclear medicine diagnosis devices and will be used and employed widely in this field.

### REFERENCE SIGNS LIST

31 bed
32 data processing device
33 display
101A, 101B detector (semiconductor radiation detector)
101a, 101b, 101c, 101d detection unit (channel)
111 semiconductor crystal
112 first electrode (anode electrode, cathode electrode)
113, 113A, 113B, 113C, 113D second electrode (cathode electrode, anode electrode)
114 side passivation layer
115 inter-divided-electrode passivation layer
300A, 300B radiation detection circuit
301A, 301B unit radiation detector circuit
311 first direct current power source
312 second direct current power source
313, 314 resistor
315 first photoMOS relay
316 second photoMOS relay
317 switch controller
318 first current regulative diode
319 second current regulative diode
320 smoothing capacitor
321 bleeder resistor
322 coupling capacitor
323 amplifier
324 polarity unifying controller
361 current regulative device
416, 417 measurement downtime
600 SPECT imaging device (nuclear medicine diagnosis device)
601A, 601B radiation detection block (camera unit)
602, 702 measurement region
603, 703 image information creating device
606 rotative supporter (camera revolving pedestal)
611 radiation measurement unit
612 interconnection substrate
613 light/electromagnetic shield
614 collimator
615 unit supporting member
700 PET imaging device (nuclear medicine diagnosis device)
701 imaging device (camera unit)
D accumulation section
H subject
K region separated with collimator
P printed substrate (interconnection substrate)

1. A semiconductor radiation detector comprising a thallium bromide semiconductor crystal sandwiched between a cathode electrode and an anode electrode,
wherein a remaining surface, among surfaces of the semiconductor crystal, which is other than a surface covered with the cathode or anode electrode, is covered with any one of two materials, that is, fluoride of thallium and chloride of thallium, or with a mixture of any one of the two materials and bromide of thallium.

2. The semiconductor radiation detector according to claim 1, wherein a plurality of detectors is disposed therein, the detector including two or more of the cathode or anode electrodes which are disposed on one surface of the semiconductor crystal and form separate channels.

3. The semiconductor radiation detector according to claim 1, wherein the cathode electrode and the anode electrode include at least one metal selected from gold, platinum, and palladium.

4. The semiconductor radiation detector according to claim 2, wherein the cathode electrode and the anode electrode include at least one metal selected from gold, platinum, and palladium.

5. A nuclear medicine diagnosis device employing the semiconductor radiation detector according to claim 1, the nuclear medicine diagnosis device comprising:
a camera unit having an interconnection substrate to which a plurality of the semiconductor radiation detectors is attached;
a camera revolving pedestal configured to revolve the camera unit in a circumferential direction of a measurement region into which a bed supporting a subject is inserted; and
an image information creating device configured to create an image from obtained information based on radiation detection signals output from the plurality of semiconductor radiation detectors in the camera unit.

6. A nuclear medicine diagnosis device employing the semiconductor radiation detector according to claim 1, the nuclear medicine diagnosis device comprising:
a camera unit in which a plurality of interconnection substrates each having a plurality of the semiconductor radiation detectors is disposed in a circumferential direction so as to encircle a measurement region into which a bed supporting a subject is inserted; and
an image information creating device connected to the interconnection substrates of the camera unit with a signal wire, and configured to create an image from obtained information based on radiation detection signals output from the plurality of semiconductor radiation detectors.
